(19) United States
(12) Patent Application Publication
(10) Pub. No.: US 2019/0355452 A1
(43) Pub. Date: Nov. 21, 2019

(54) METHOD AND SYSTEM TO SAFEGUARD RELEASE OF MEDICAL RECORDS

(71) Applicant: Mizan Rahman, Atlanta, GA (US)

(72) Inventor: Mizan Rahman, Atlanta, GA (US)

(21) Appl. No.: 16/416,235

(22) Filed: May 19, 2019

Related U.S. Application Data
(60) Provisional application No. 62/673,933, filed on May 20, 2018.

(51) Int. Cl. G16H 10/60; G16H 50/30; G16H 80/00; G16H 50/20

(52) U.S. Cl. CPC G16H 10/60; G16H 50/30; G16H 40/20; G16H 50/20; G16H 80/00

(57) ABSTRACT

The present invention discloses a system and method for sharing medical records of a user from a first entity to a second entity. The method includes receiving a data release request from the user to release medical records of the user, determining whether photo of the user is available on the medical records, determining whether a medical application is installed on a communication device of the user, sending a push notification to the communication device, opening the medical application on the communication device upon receiving a trigger signal from the user, activating an image capturing sensor of the communication device upon opening of the medical application, capturing the photo of the user through the image capturing sensor, determining whether the user is an authorized user by analyzing the captured photo, and sharing medical records of the user from the first entity to the second entity.

### CROSS-REFERENCE TO RELATED PATENT DOCUMENTS

This patent application claims the benefit of priority of U.S. Provisional Application No. 62/673,933 entitled “METHOD AND SYSTEM TO SAFEGUARD RELEASE OF MEDICAL RECORDS,” filed May 20, 2018, which are hereby incorporated herein by reference in its entirety.

### FIELD OF THE INVENTION

The present invention relates generally to medical records, and, more particularly, to system and method for safeguarding release of medical records of a user.

### BACKGROUND

In healthcare industry, patient's record is the main tool for diagnosing a patient. It is believed that every year a significant number of people die due to medical errors by highly qualified, skilled and trained physicians and hospitals. Most of these medical errors result from physicians and hospitals due to not having the patient's full medical history at the time of treatment. Hospitals and doctors are being urged to convert their patients' hardcopy medical records into an electronic format in an effort to improve health care and save lives. The medical and health care records of an individual are highly personal documents often containing private, sensitive information.

The release of medical information for commercial use is strictly regulated by state and federal law. Thus, medical records are becoming more generally available in electronic form, but electronic signatures authorizing release of such records to others are not easily accessible today. Because such records are highly sensitive, the records are protected by laws requiring patient consent prior to release or disclosure to others.

Health care providers are legally obligated to protect patient data and patient privacy. When a patient goes from one hospital to another or from one physician to the other, they need to bring the health record from the previous providers. As standard process, patient signs a medical data release form and send to his/her previous health care service provider. Upon receiving the release document, provider sends the data to either new provider or to the patient. However, this relaxed process opens up a serious security hole. There is evidence that health care provider sometime sends information of the wrong patient to new providers or even to the patient. Criminals with knowledge about such security flaw, could send a medical data release form of a person of interest and end up receiving the intended information since there is no solid validation method of the medical data release process. The current invention proposes a method and system to safeguard release of patient medical data to avoid criminals or even a wrong person accidently receiving someone else's medical record.

In light of the foregoing, there exists a need for a technical and reliable solution that solves the above-mentioned problems and presents an improved method and system to safeguard release of medical records of a user.

### BRIEF SUMMARY

It is an objective of the present invention to provide a method and system to safeguard release of patient medical data to avoid criminals or even a wrong person accidently receiving someone else's medical record.

It is another objective of the present invention to provide a system and method to authenticate a user before releasing medical data of the user from a first entity to a second entity.

In an embodiment, the present invention discloses a method for sharing medical records of a user from the first entity to the second entity. The method includes receiving a data release request from the user to release medical records of the user, determining whether photo of the user is available on the medical records, determining whether a medical application is installed on a communication device of the user, sending a push notification to the communication device, opening the medical application on the communication device upon receiving a trigger signal from the user, activating an image capturing sensor of the communication device upon opening of the medical application, capturing the photo of the user through the image capturing sensor, determining whether the user is an authorized user by analyzing the captured photo, and sharing medical records of the user from the first entity to the second entity.

These and other features and advantages of the present invention will become apparent from the detailed description below, in light of the accompanying drawings.

### BRIEF DESCRIPTION OF THE ACCOMPANYING DRAWINGS

The novel features which are believed to be characteristic of the present invention, as to its structure, organization, use and method of operation, together with further objectives and advantages thereof, will be better understood from the following drawings in which a presently preferred embodiment of the invention will now be illustrated by way of various examples. It is expressly understood, however, that the drawings are for the purpose of illustration and description only and are not intended as a definition of the limits of the invention. Embodiments of this invention will now be described by way of example in association with the accompanying drawings in which:

FIG. 1 illustrates a system for sharing medical records of a user from a first entity to a second entity, according to an exemplary embodiment of the present invention;

FIGS. 2A-2C is a flowchart illustrating a method for authenticating the user before releasing the medical records of the user from the first entity to the second entity, according to an exemplary embodiment of the present invention;

FIG. 3 is a flowchart illustrating a method for sharing medical records of the user from the first entity to the second entity, according to an exemplary embodiment of the present invention;

FIG. 4 illustrates a block diagram of a medical record sharing platform, according to an exemplary embodiment of the present invention; and

FIG. 5 is a block diagram that illustrates a system architecture of a computer system for sharing medical records of the user from the first entity to the second entity, according to an exemplary embodiment of the present invention.

Further areas of applicability of the present invention will become apparent from the detailed description provided hereinafter. It should be understood that the detailed description of exemplary embodiments is intended for illustration purposes only and is, therefore, not intended to necessarily limit the scope of the invention.

### DETAILED DESCRIPTION

As used in the specification and claims, the singular forms “a”, “an” and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention. There may be additional components described in the foregoing application that are not depicted on one of the described drawings. In the event such a component is described, but not depicted in a drawing, the absence of such a drawing should not be considered as an omission of such design from the specification.

Before describing the present invention in detail, it should be observed that the present invention utilizes a combination of components, which constitutes a system to safeguard release of medical records. Accordingly, the components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

Techniques consistent with the present invention provide, among other features, a method and a system to safeguard release of medical records. The release of patient medical data is safeguarded to avoid criminals or even a wrong person accidently receiving someone else's medical record.

Unless stated otherwise, terms such as “first” and “second” are used to arbitrarily distinguish between the elements or entities. Thus, these terms are not necessarily intended to indicate temporal or other prioritization of such elements or priorities. While various exemplary embodiments of the disclosed systems and methods have been described above, it should be understood that they have been presented for purposes of example only, and not limitations. It is not exhaustive and does not limit the invention to the precise form disclosed. Modifications and variations are possible in light of the above teachings or may be acquired from practicing of the invention, without departing from the breadth or scope.

The method and system of the present invention will now be described with reference to the accompanying drawings, which should be regarded as merely illustrative without restricting the scope and ambit of the present invention.

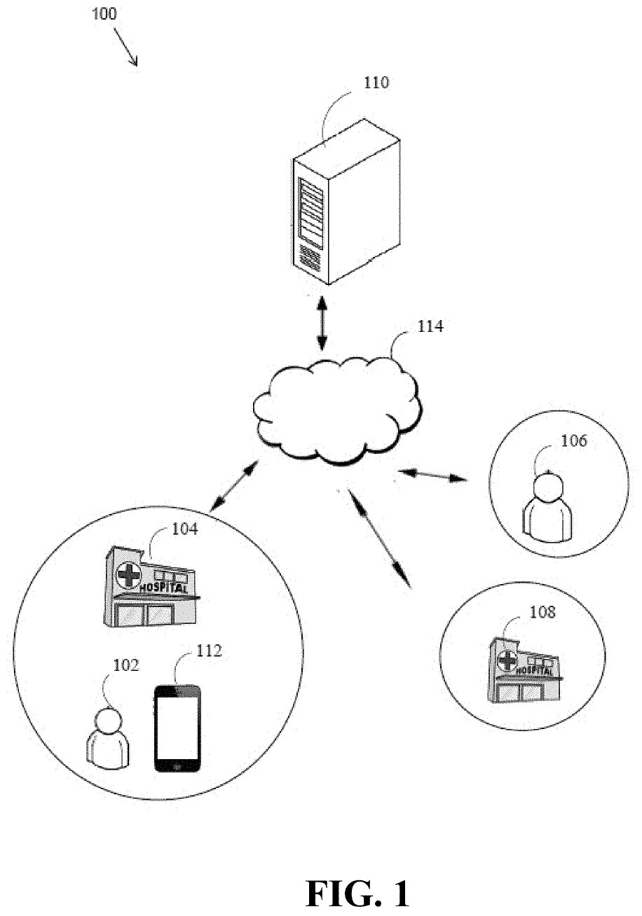

Referring now to figures, FIG. 1 illustrates a system 100 for sharing medical records of a user from a first entity to a second entity, according to an exemplary embodiment of the present invention. The system 100 includes a user 102, a first entity 104, a communication device 112 associated with the user 102, a communication network 114, a medical record sharing platform 110, an authorized person 106, and a second entity 108. Examples of the first entity 104 or the second entity 108 may include, but are not limited to, a hospital, a medical, a clinic, a nursing home, and a healthcare organization. In context of the present invention, the user 102 is preferably a patient. Further, in context of the present invention, the authorized person 106 is a person who is authorized to check the medical records of the user 102. Examples of the authorized person 106 may include, but are not limited to, a physician, a doctor, a radiologist, a medical specialist, and a clinician. The medical records may include medical history of the user 102. In clinical medicine, the patient's (e.g., the user 102) past and present which may contain relevant information bearing on their health past, present, and future. The medical history, being an account of all medical events and problems a person has experienced is an important tool in the management of the patient. For example, the medical records may include name, address, photo, illness records, payment records, insurance records, or the like of the patient.

Consider a scenario where the user 102 requested the first entity 104 to release his/her medical records to the second entity 108 or to the authorized person 106. After receiving the request from the user 102, the first entity 104 determines whether an enrolment photo of the user 102 and/or other stored photos of the user 102 is available on the medical record sharing platform 110 or is not available on the medical record sharing platform 110.

In an embodiment, the medical record sharing platform 110 may include suitable logic, circuitry, interfaces, and/or code, executable by the circuitry, that may be configured to perform one or more operations. The medical record sharing platform 110 may be a computing device or application, which may include a software framework, that may be configured to create the server implementation and perform the various operations. The medical record sharing platform 110 may be realized through various web-based technologies, such as, but not limited to, a Java web-framework, a .NET framework, a professional hypertext preprocessor (PHP) framework, a python framework, or any other web-application framework. The medical record sharing platform 110 may also be realized as a machine-learning model that implements any suitable machine-learning techniques, statistical techniques, or probabilistic techniques. Examples of such techniques may include expert systems, fuzzy logic, support vector machines (SVM), Hidden Markov models (HMMs), greedy search algorithms, rule-based systems, Bayesian models (e.g., Bayesian networks), neural networks, decision tree learning methods, other non-linear training techniques, data fusion, utility-based analytical systems, or the like. Examples of the medical record sharing platform 110 may include, but are not limited to, a personal computer, a laptop, or a network of computer systems.

In another embodiment, the medical record sharing platform 110 can be distributed to the user 102 as a web based platform for a web browser installed on the communication device 112. In another embodiment, the medical record sharing platform 110 may be an add-on to the web browser installed on the communication device 112. In another embodiment, the medical record sharing platform 110 can be an application installed on the communication device 112. If it is determined by the medical record sharing platform 110 that the photo of the user 102 is available, then the medical record sharing platform 110 determines whether a medical application, in communication with the medical record sharing platform 110, is installed on the communication device 112 or not.

In context of the present invention, the communication device 112 refers to an electronic device that can be used to communicate over the communication network 114. Examples of the communication device 112 may include, but are not limited to, a cell phone, a smart phone, a cellular phone, a cellular mobile phone, a personal digital assistant (PDA), a wireless communication terminal, a laptop, PC, and a tablet computer.

Various components of the system 100 are capable of connecting to the Internet. It should be noted that the term “Internet” is intended to encompass similar systems as well (i.e., World Wide Web or “www”) comprising the capability to communicate and access information through the communication network 114. Examples of types of the communication network 114 may include, but are not limited to, a local area network, a wide area network, a radio network, a virtual private network, an internet area network, a metropolitan area network, a satellite network, Wi-Fi, Bluetooth Low energy, a wireless network, and a telecommunication network. Examples of the telecommunication network include, but are not be limited to a global system for mobile communication (GSM) network, a general packet radio service (GPRS) network, third Generation Partnership Project (3GPP), an enhanced data GSM environment (EDGE) and a Universal Mobile Telecommunications System (UMTS). The present invention should not be limited in its communication nomenclature.

If it is determined by the medical record sharing platform 110 that the medical application is installed on the communication device 112, then the medical record sharing platform 110 determines whether the medical application is capable of remote user authentication or not. If it is determined by the medical record sharing platform 110 that the medical application is capable of remote user authentication, then the medical record sharing platform 110 sends a push notification to the communication device 112 to perform a user photo capture (for example, capturing an image of a user such as the user 102) in order to verify the medical record release request. Further, the medical record sharing platform 110 activate an image capturing sensor (e.g., a camera) upon receiving an activation signal (e.g., clicking the push notification) from the user 102 to capture the photo of the user 102.

In an embodiment, the image capturing sensor can be activated manually by the user 102. Further, the medical record sharing platform 110 determines whether the user 102 is an authorized user by comparing the captured photo of the user 102 with the stored photo of the user 102. If there is a match between the captured photo and the stored photo of the user 102, then the medical record sharing platform 110 sends authentication success message to the user 102 and release the medical record of the user 102 to the second entity 108. If there is no match between the captured photo and the stored photo of the user 102, then the medical record sharing platform 110 sends authentication unsuccess message to an administrator of the medical record sharing platform 110.

Further, if it is determined by the medical record sharing platform 110 that the medical application is not installed on the communication device 112, then the medical record sharing platform 110 retrieve contact details of the user 102. In an embodiment, the contact details include an email id, a physical address, name, age, gender, contact number, street number, and the like. Further, the medical record sharing platform 110 sends a message (e.g., Short message service)/email including a weblink to the user 102. If it is determined by the medical record sharing platform 110 that the user 102 has opened the weblink, then the medical record sharing platform 110 receive verification details from the user 102. In an embodiment, the verification details include a code, a number, a letter, and the like. Upon receiving the verification details, the medical record sharing platform 110 activate the image capturing sensor (e.g., a camera) upon receiving an activation signal (e.g., clicking the push notification) from the user 102 to capture the photo of the user 102.

Further, the medical record sharing platform 110 determines whether the user 102 is an authorized user by comparing the captured photo with the stored photo of the user 102. If there is a match between the captured photo and the stored photo of the user 102, then the medical record sharing platform 110 sends authentication success message to the user 102 and release the medical record of the user 102 to the second entity 108. If there is no match between the captured photo and the stored photo of the user 102, then the medical record sharing platform 110 sends authentication unsuccess message to the administrator of the medical record sharing platform 110.

Further, if it is determined by the medical record sharing platform 110 that the medical application is installed on the communication device 112, however, the medical application is not capable of remote user authentication, then the medical record sharing platform 110 retrieve contact details of the user 102. In an embodiment, the contact details include an email id, a physical address, name, age, gender, contact number, street number, and the like. Further, the medical record sharing platform 110 sends a message (e.g., Short message service)/email with a weblink to the user 102. If it is determined by the medical record sharing platform 110 that the user 102 has opened the weblink, then the medical record sharing platform 110 receive verification details from the user 102. In an embodiment, the verification details include a code, a number, a letter, and the like. Upon receiving the verification details, the medical record sharing platform 110 activate the image capturing sensor (e.g., a camera) upon receiving an activation signal (e.g., clicking the push notification) from the user 102 to capture the photo of the user 102. Further, the medical record sharing platform 110 determines whether the user 102 is an authorized user by comparing the captured photo with the stored photo of the user 102. If there is a match between the captured photo and the stored photo of the user 102, then the medical record sharing platform 110 sends authentication success message to the user 102 and release the medical record of the user 102 to the second entity 108. If there is no match between the captured photo and the stored photo of the user 102, then the medical record sharing platform 110 sends authentication unsuccess message to the administrator of the medical record sharing platform 110.

Further, if it is determined by the medical record sharing platform 110 that the photo of the user 102 is unavailable, then the medical record sharing platform 110 sends a message to an authority of the first entity 104 to manually verify the user 102 and release the medical records of the user 102 after successful verification.

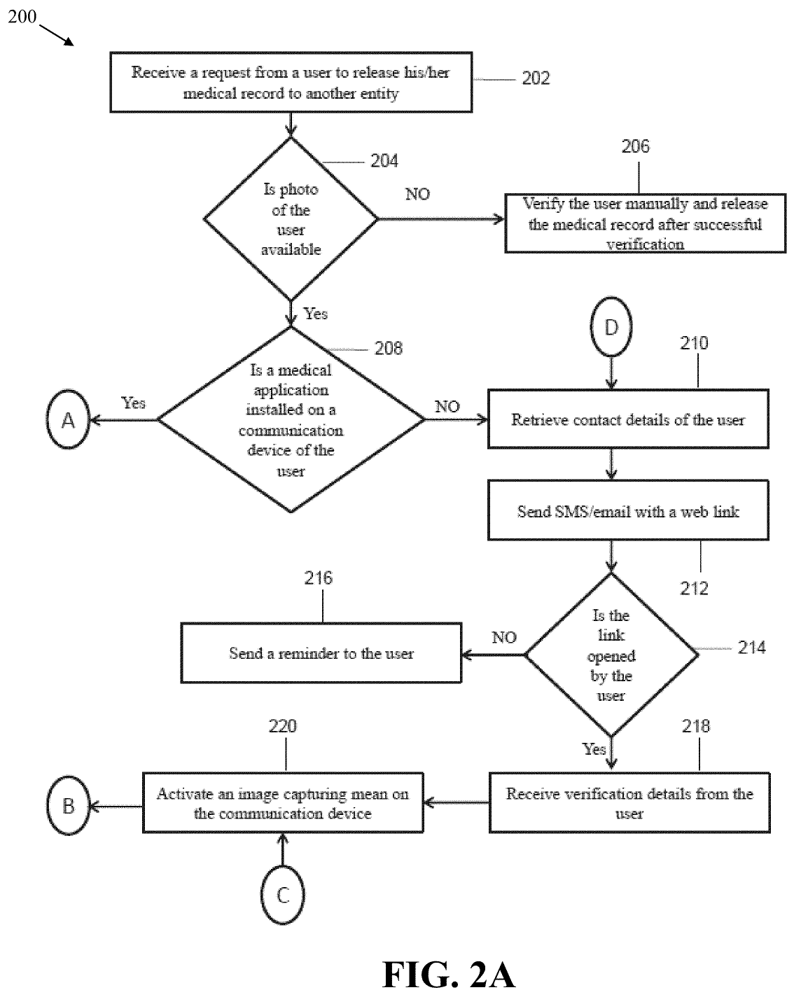

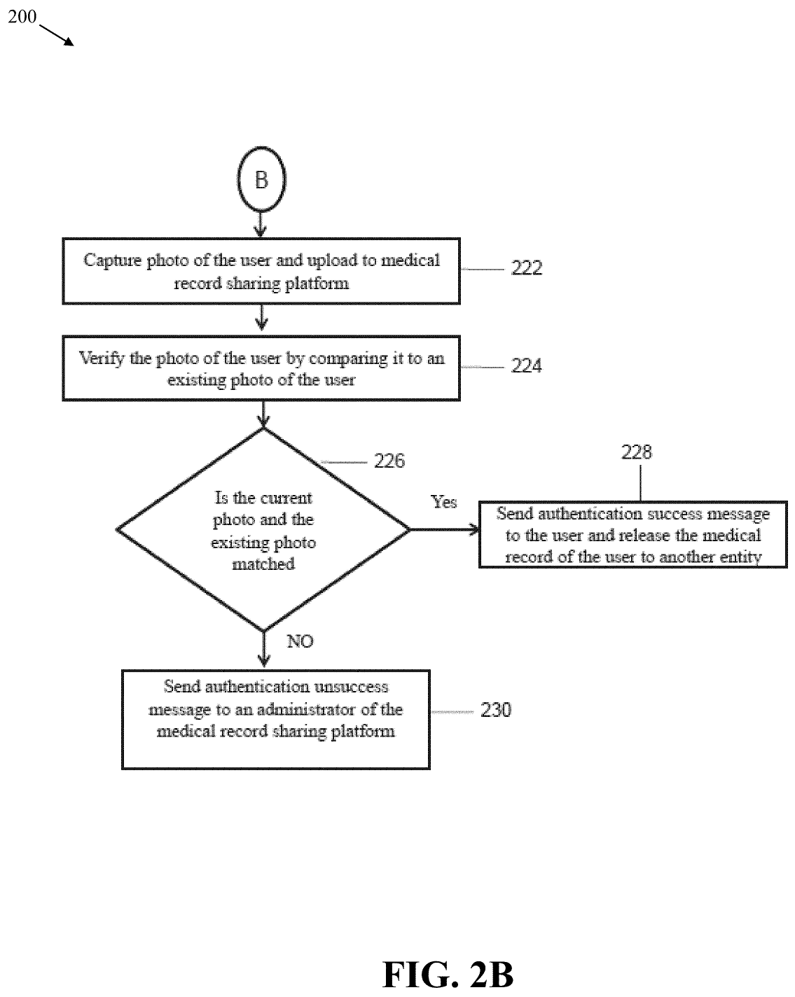

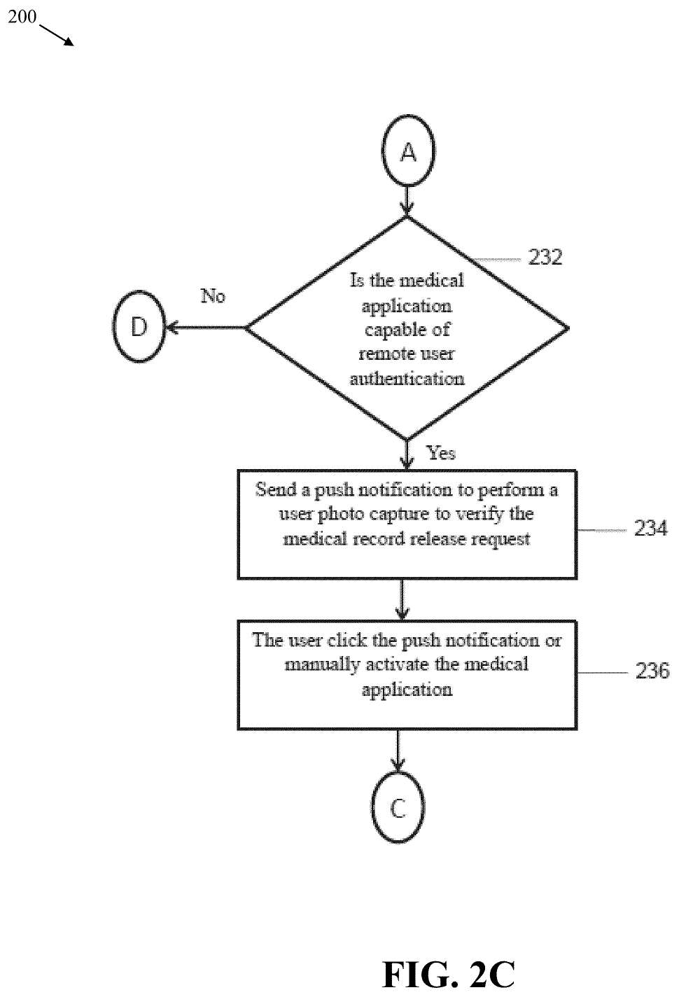

FIG. 2A-2C is a flowchart 200 illustrating a method for authenticating the user 102 before releasing the medical records of the user 102 from the first entity 104 to the second entity 108 or to the authorized person 106, according to an exemplary embodiment of the present invention.

At step 202, the method includes receiving the request from the user 102 to release his/her medical records from the first entity 104 to the second entity 108 or to the authorized person 106. The method allows the medical record sharing platform 110 to receive the request from the user 102 to release his/her medical records from the first entity 104 to the second entity 108 or to the authorized person 106. In other way round, the medical record sharing platform 110 may be configured to receive the request from the user 102 to release his/her medical records from the first entity 104 to the second entity 108 or to the authorized person 106.

At step 204, the method determines whether the enrolment photo of the user 102 and/or other stored photos of the user 102 is available with the medical record sharing platform 110 or not. The method allows the medical record sharing platform 110 to determine whether the enrolment photo of the user 102 and/or other stored photos of the user 102 is available or not. In other way round, the medical record sharing platform 110 may be configured to determine whether the enrolment photo of the user 102 and/or other stored photos of the user 102 is available or not. If it is determined, at step 204, that the enrolment photo of the user 102 and/or other stored photos of the user 102 is not available, then, at step 206, the method sends the message to an authority of the first entity 104 to manually verify the user 102 and release the medical records of the user 102 after successful verification. The method allows the medical record sharing platform 110 to send the message to the authority of the first entity 104 to manually verify the user 102 and release the medical records of the user 102 after successful verification. In other way round, the medical record sharing platform 110 may be configured to send the message to the authority of the first entity 104 to manually verify the user 102 and release the medical records of the user 102 after successful verification.

If it is determined, at step 204, that the enrolment photo of the user 102 and/or other stored photos of the user 102 is available, then, at step 208, the method determines whether the medical application, in communication with the medical record sharing platform 110, is installed on the communication device 112 or not.

If it is determined, at step 208, that the medical application is not installed on the communication device 112, then, at step 210, the method retrieves the contact details of the user 102. The contact details may include an email id, a physical address, name, age, gender, contact number, street number, and the like. Further, at step 212, the method sends the message (e.g., Short message service)/email including the weblink to the user 102.

Further, at step 214, the method determines whether the user 102 has opened the weblink or not. If it is determined, at step 214, that the user 102 has not opened the weblink, then, at step 216, the method includes sending a reminder to the user 102. The method allows the medical record sharing platform 110 to send the reminder to the user 102. In other way round, the medical record sharing platform 110 may be configured to send the reminder to the user 102. If it is determined, at step 214, that the user 102 has opened the weblink, then, at step 218, the method includes receiving the verification details from the user 102. The method allows the medical record sharing platform 110 to receive the verification details from the user 102. In other way round, the medical record sharing platform 110 may be configured to receive the verification details from the user 102. Upon receiving the verification details, at step 220, the method includes activating the image capturing sensor (e.g., a camera) to capture the photo of the user 102. The method allows the medical record sharing platform 110 to activate the image capturing sensor (e.g., a camera) to capture the photo of the user 102. In other way round, the medical record sharing platform 110 may be configured to activate the image capturing sensor (e.g., a camera) to capture the photo of the user 102.

Further, at step 222, the method includes capturing the photo of the user 102 and uploading to the medical record sharing platform 110. The method allows the medical record sharing platform 110 to capture the photo of the user 102 and uploading to the database (not shown). In other way round, the medical record sharing platform 110 may be configured to capture the photo of the user 102 by means of the image capturing sensor and uploading to the database. Further, at step 224, the method includes verifying the photo of the user 102 by comparing the captured photo with previously stored photo of the user 102. The method allows the medical record sharing platform 110 to verify the photo of the user 102 by comparing the captured photo with previously stored photo of the user 102. In other way round, the medical record sharing platform 110 may be configured to verify the photo of the user 102 by comparing the captured photo with previously stored photo of the user 102

Further, at step 226, the method includes step of determining whether there is a match between the captured photo with previously stored photo of the user 102 or not. The method allows the medical record sharing platform 110 to determine whether there is a match between the captured photo and previously stored photo of the user 102 or not. In other way round, the medical record sharing platform 110 may be configured to determine whether there is a match between the captured photo and previously stored photo of the user 102 or not. If it is determined, at step 226, that there is a match between the captured photo and previously stored photo of the user 102, then, at step 228, the method includes a step of sending an authentication success message to the user 102 and releases the medical records of the user 102 to the second entity 108. If it is determined, at step 226, that there is no match between the captured photo and previously stored photo of the user 102, then, at step 230, the method includes a step of sending an authentication unsuccess message to the administrator of the medical record sharing platform 110.

If it is determined, at step 208, that the medical application is installed on the communication device 112, then, at step 232, the method includes determining whether the medical application is capable of remote user authentication or not. The method allows the medical record sharing platform 110 to determine whether the medical application is capable of remote user authentication or not. In other way round, the medical record sharing platform 110 may be configured to determine whether the medical application is capable of remote user authentication or not. If it is determined, at step 232, that the medical application is capable of remote user authentication, then, at step 234, the method includes a step of sending the push notification to perform the user photo capture to verify the medical record release request. The method allows the medical record sharing platform 110 to send the push notification to perform the user photo capture to verify the medical record release request. Further, at step 236, the method includes activating the medical application upon receiving an activation signal (e.g., clicking the push notification or manual activation) from the user 102. The method allows the medical record sharing platform 110 to activate the medical application upon receiving an activation signal (e.g., clicking the push notification or manual activation) from the user 102. Upon activating the medical application, at step 220, the method includes activating the image capturing sensor (e.g., a camera) to capture the photo of the user 102. The method allows the medical record sharing platform 110 to activate the image capturing sensor (e.g., a camera) to capture the photo of the user 102. In other way round, the medical record sharing platform 110 may be configured to activate the image capturing sensor (e.g., a camera) to capture the photo of the user 102.

Further, at step 222, the method includes capturing the photo of the user 102 and uploading to the medical record sharing platform 110. The method allows the medical record sharing platform 110 to capture the photo of the user 102 and uploading to the database (not shown). Further, at step 224, the method includes verifying the photo of the user 102 by comparing the captured photo with previously stored photo of the user 102. The method allows the medical record sharing platform 110 to verify the photo of the user 102 by comparing the captured photo with previously stored photo of the user 102. In other way round, the medical record sharing platform 110 may be configured to verify the photo of the user 102 by comparing the captured photo with previously stored photo of the user 102.

Further, at step 226, the method includes determining whether there is a match between the captured photo with previously stored photo of the user 102 or not. The method allows the medical record sharing platform 110 to determine whether there is a match between the captured photo and previously stored photo of the user 102 or not. If it is determined, at step 226, that there is a match between the captured photo and previously stored photo of the user 102, then, at step 228, the method includes sending an authentication success message to the user 102 and release the medical records of the user 102 to the second entity 108. If it is determined, at step 226, that there is no match between the captured photo and previously stored photo of the user 102, then, at step 230, the method includes sending an authentication unsuccess message to the administrator of the medical record sharing platform 110.

If it is determined, at step 232, that the medical application is not capable of remote user authentication, then, at step 210, the method includes retrieving the contact details of the user 102. The method allows the medical record sharing platform 110 to retrieve the contact details of the user 102. Further, at step 212, the method includes sending the message (e.g., Short message service)/email including the weblink to the user 102. The method allows the medical record sharing platform 110 to send the message (e.g., Short message service)/email including the weblink to the user 102.

Further, at step 214, the method includes determining whether the user 102 has opened the weblink or not. The method allows the medical record sharing platform 110 to determine whether the user 102 has opened the weblink or not. If it is determined, at step 214, that the user 102 has not opened the weblink, then, at step 216, the method includes sending a reminder to the user 102. The method allows the medical record sharing platform 110 to send the reminder to the user 102. If it is determined, at step 214, that the user 102 has opened the weblink, then, at step 218, the method includes receiving the verification details from the user 102. The method allows the medical record sharing platform 110 to receive the verification details from the user 102. Upon receiving the verification details, at step 220, the method includes activating the image capturing sensor (e.g., a camera) to capture the photo of the user 102. The method allows the medical record sharing platform 110 to activate the image capturing sensor (e.g., a camera) to capture the photo of the user 102.

Further, at step 222, the method includes capturing the photo of the user 102 and uploading to the medical record sharing platform 110. The method allows the medical record sharing platform 110 to capture the photo of the user 102 and uploading to the database (not shown). Further, at step 224, the method includes verifying the photo of the user 102 by comparing the captured photo with previously stored photo of the user 102. The method allows the medical record sharing platform 110 to verify the photo of the user 102 by comparing the captured photo with previously stored photo of the user 102.

Further, at step 226, the method includes determining whether there is a match between the captured photo with previously stored photo of the user 102 or not. The method allows the medical record sharing platform 110 to determine whether there is a match between the captured photo and previously stored photo of the user 102 or not. If it is determined, at step 226, that there is a match between the captured photo and previously stored photo of the user 102, then, at step 228, the method includes sending an authentication success message to the user 102 and releases the medical records of the user 102 to the second entity 108. If it is determined, at step 226, that there is no match between the captured photo and previously stored photo of the user 102, then, at step 230, the method includes sending an authentication unsuccess message to the administrator of the medical record sharing platform 110.

The various actions, acts, blocks, steps, or the like in the flow diagram may be performed in the order presented, in a different order or simultaneously. Further, in some embodiments, some of the actions, acts, blocks, steps, or the like may be omitted, added, modified, skipped, or the like without departing from the scope of the present invention.

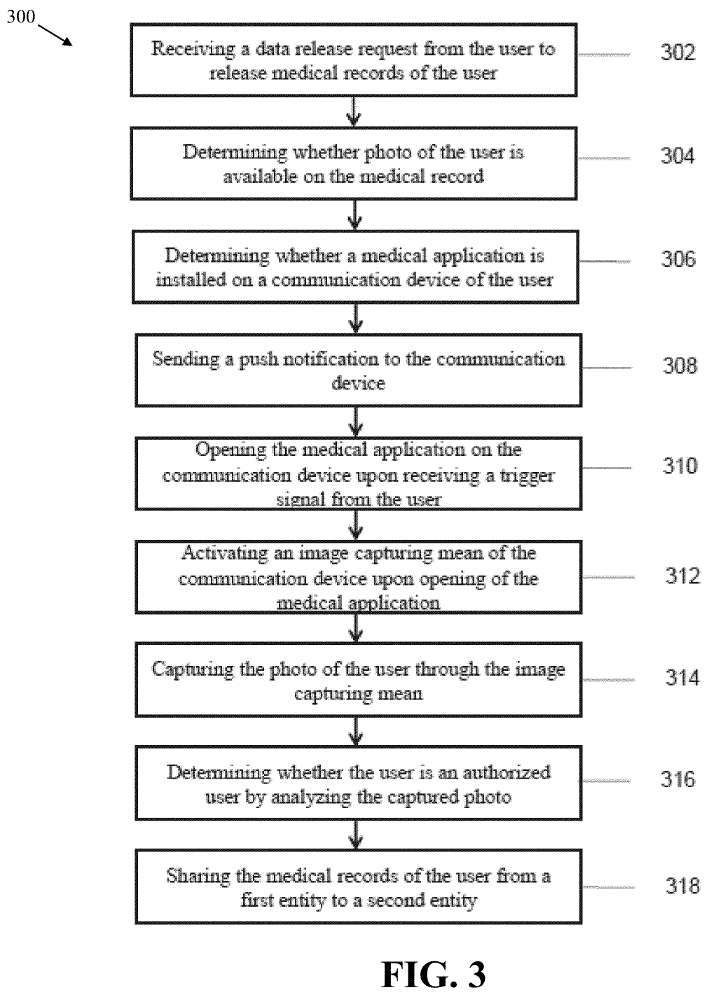

FIG. 3 is a flowchart 300 illustrating a method for sharing medical records of the user 102 from the first entity 104 to the second entity 108, according to an exemplary embodiment of the present invention. At step 302, the method includes receiving the data release request from the user 102 (for example, from the communication device 112 of the user 102) to release medical records of the user 102. The method allows the medical record sharing platform 110 to receive the data release request from the user 102 for example, from the communication device 112 of the user 102) to release medical records of the user 102. In other way round, the medical record sharing platform 110 may be configured to receive the data release request from the user 102 for example, from the communication device 112 of the user 102) to release medical records of the user 102.

At step 304, the method includes determining whether the photo of the user 102 is available on the medical record or not. The method allows the medical record sharing platform 110 to determine whether the photo of the user 102 is available on the medical record or not. In other way round, the medical record sharing platform 110 may be configured to determine whether the photo of the user 102 is available on the medical record or not. At step 306, the method includes determining whether the medical application is installed on the communication device 112 of the user 102 or not. The method allows the medical record sharing platform 110 to determine whether the medical application is installed on the communication device 112 of the user 102 or not. In other way round, the medical record sharing platform 110 may be configured to determine whether the medical application is installed on the communication device 112 of the user 102 or not.

At step 308, the method includes sending the push notification to the communication device 112. The method allows the medical record sharing platform 110 to send the push notification to the communication device 112. In other way round, the medical record sharing platform 110 may be configured to send the push notification to the communication device 112. At step 310, the method includes opening the medical application on the communication device 112 upon receiving the trigger signal from the user 102. The method allows the medical record sharing platform 110 to open the medical application on the communication device 112 upon receiving the trigger signal from the user 102. In other way round, the medical record sharing platform 110 may be configured to open the medical application on the communication device 112 upon receiving the trigger signal from the user 102.

At step 312, the method includes activating the image capturing sensor of the communication device 112 upon opening of the medical application. The method allows the medical record sharing platform 110 to activate the image capturing sensor of the communication device 112 upon opening of the medical application. In other way round, the medical record sharing platform 110 may be configured to activate the image capturing sensor of the communication device 112 upon opening of the medical application.

At step 314, the method includes capturing the photo of the user 102 through the image capturing sensor. The method allows the medical record sharing platform 110 to capture the photo of the user 102 through the image capturing sensor. In other way round, the medical record sharing platform 110 may be configured to capture the photo of the user 102 through the image capturing sensor. At step 316, the method includes determining whether the user 102 is an authorized user by analyzing the captured photo. The method allows the medical record sharing platform 110 to determine whether the user 102 is an authorized user by analyzing the captured photo. In other way round, the medical record sharing platform 110 may be configured to determine whether the user 102 is an authorized user by analyzing the captured photo. At step 318, the method includes sharing the medical records of the user 102 from the first entity 104 to the second entity 108. The method allows the medical record sharing platform 110 to share the medical records of the user 102 from the first entity 104 to the second entity 108. In other way round, the medical record sharing platform 110 may be configured to share the medical records of the user 102 from the first entity 104 to the second entity 108.

The various actions, acts, blocks, steps, or the like in the flow diagram may be performed in the order presented, in a different order or simultaneously. Further, in some embodiments, some of the actions, acts, blocks, steps, or the like may be omitted, added, modified, skipped, or the like without departing from the scope of the invention.

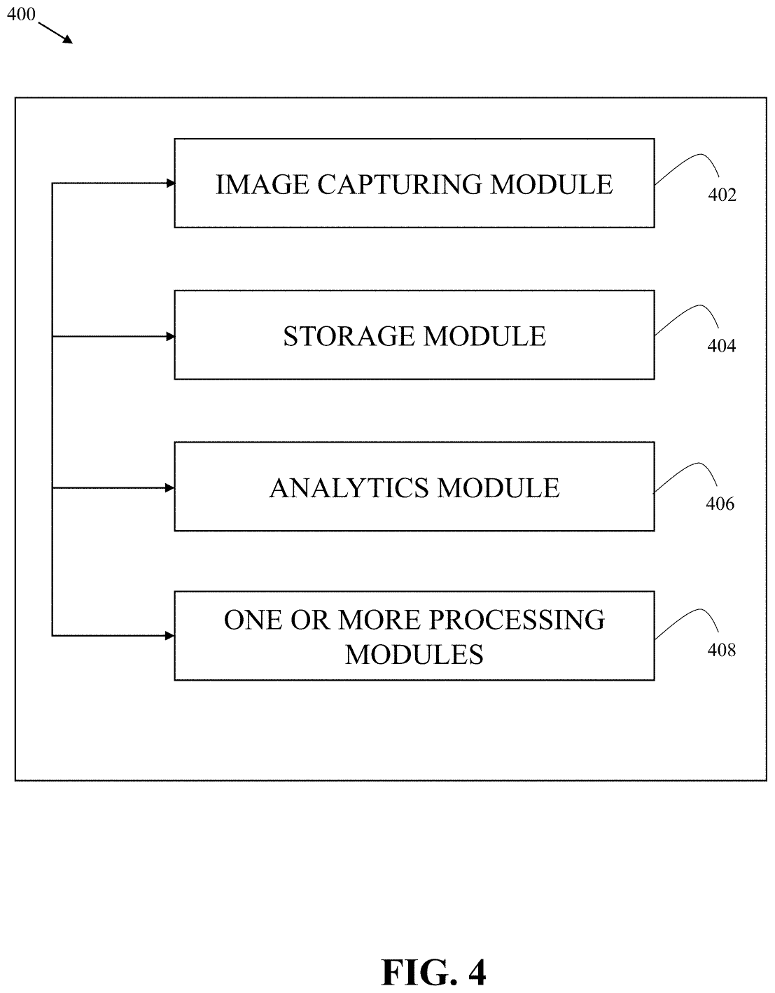

FIG. 4 illustrates a block diagram 400 of the medical record sharing platform 110, according to an exemplary embodiment of the present invention. In an embodiment, the medical record sharing platform 110 includes an image capturing module 402, a storage module 404, an analytics module 406, and one or more processing module 408.

The image capturing module 402 may include suitable logic, circuitry, interfaces, and/or code, executable by the circuitry, that may be configured to perform one or more operations. The image capturing module 402 is configured to capture the image (e.g., the facial photo) of the user 102. Further, the image capturing module 402 sends the captured photo to the analytics module 406 for further analysis.

The storage module 404 may include suitable logic, circuitry, interfaces, and/or code, executable by the circuitry, that may be configured to perform one or more operations. The storage module 404 is configured to store data related to both the user 102 and the registered user. In an embodiment, the storage module 404 is a multi-tier storage system. In another embodiment, the storage module 404 stores the information in an encrypted format. In yet another embodiment, the storage module 404 stores the information in an indexed format. The storage module 404 facilitates storage, retrieval, modification, and deletion of data in conjunction with various data-processing operations. Storage module information may be retrieved through queries using keywords and sorting commands, in order to rapidly search, rearrange, group, and select the field.

In one embodiment, the storage module 404 is secure web servers and Hypertext Transport Protocol Secure (HTTPS) capable of supporting Transport Layer Security (TLS). Communications to and from the secure web servers may be secured using Secure Sockets Layer (SSL). An SSL session may be started by sending a request to the Web server with an HTTPS prefix in the URL. Alternatively, any known communication protocols that enable devices within a computer network to exchange information may be used. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol), POP3 (Post Office Protocol 3), SMTP (Simple Mail Transfer Protocol), IMAP (Internet Message Access Protocol), SOAP (Simple Object Access Protocol), PPP (Point-to-Point Protocol), RFB (Remote Frame buffer) Protocol.

In an embodiment, the analytics module 406 may include suitable logic, circuitry, interfaces, and/or code, executable by the circuitry, that may be configured to perform one or more operations. The analytics module 406 obtains the stored pictures of the user 102 from the storage module 404 and compare the captured photo of the user 102 and the stored photo of the user 102. If there is a match between the captured photo and the stored photo of the user 102, then the analytics module 406 sends authentication success message to the user 102 and release the medical record of the user 102 to the second entity 108. If there is no match between the captured photo and the stored photo of the user 102, then the analytics module 406 sends authentication unsuccess message to the administrator of the medical record sharing platform 110.

The one or more processing module 408 is configured to process data and images related to the drug abusers from various sources. Further, the one or more processing module 408 is also configured to process the data related to the user 102 and the registered user. The one or more processing module 408 is associated with a memory module. The memory module accessible by the one or more processing module 408 receives and stores data. The memory module may be a main memory, such as a high speed Random Access Memory (RAM), or an auxiliary storage unit, such as a hard disk, a floppy disk, or a magnetic tape drive. The memory may be any other type of memory, such as a Read-Only Memory (ROM), or optical storage media such as a videodisc and a compact disc. The one or more processing module 408 may access the memory module to retrieve data. Examples of one or more processing module 408 may include, but are not limited to, a central processing unit (CPU), a front-end processor, a microprocessor, a graphics processing unit (GPUNPU), a physics processing unit (PPU), a digital signal processor, and a network processor.

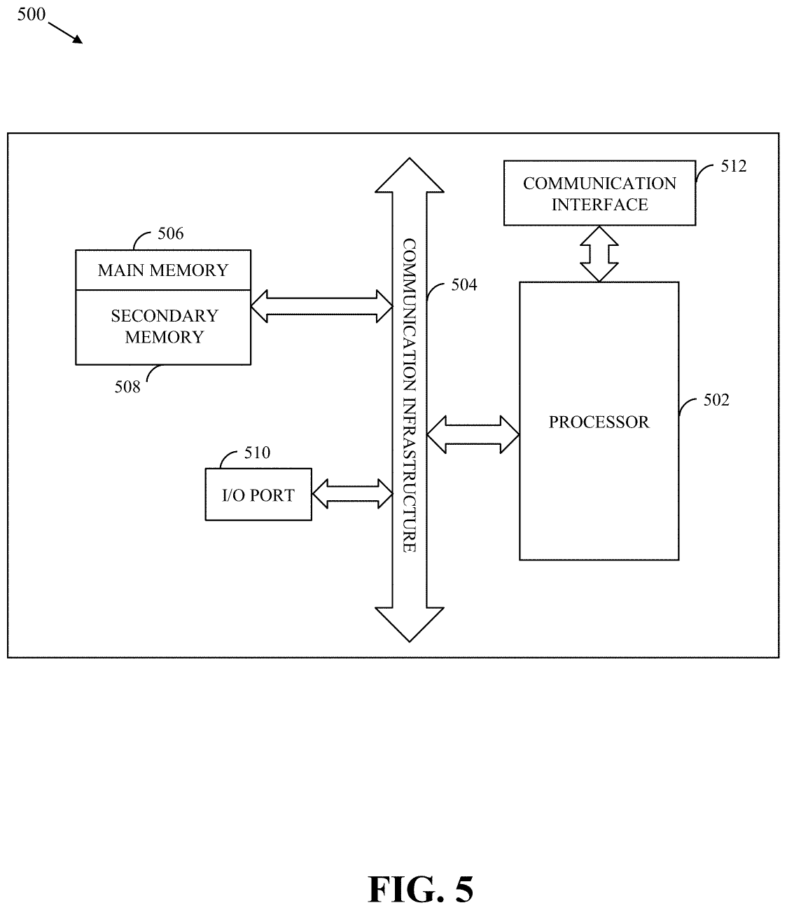

FIG. 5 is a block diagram that illustrates a system architecture of a computer system 500 for sharing medical records of the user 102 from the first entity 104 to the second entity 108, according to an exemplary embodiment of the present invention. An embodiment of the disclosure, or portions thereof, may be implemented as computer readable code on the computer system 500. In one example, various components (such as the medical record sharing platform 110) of FIG. 1 and/or FIG. 4 may be implemented in the computer system 500 using hardware, software, firmware, non-transitory computer readable media having instructions stored thereon, or a combination thereof and may be implemented in one or more computer systems or other processing systems. Hardware, software, or any combination thereof may embody modules and components used to implement the methods of FIGS. 2A-2C and 3.

The computer system 500 may include a processor 502 that may be a special purpose or a general-purpose processing device. The processor 502 may be a single processor, multiple processors, or combinations thereof. The processor 502 may have one or more processor “cores.” Further, the processor 502 may be coupled to a communication infrastructure 504, such as a bus, a bridge, a message queue, the communication network 114, multi-core message-passing scheme, and the like. The computer system 500 may further include a main memory 506 and a secondary memory 508. Examples of the main memory 506 may include RAM, ROM, and the like. The secondary memory 508 may include a hard disk drive or a removable storage drive (not shown), such as a floppy disk drive, a magnetic tape drive, a compact disc, an optical disk drive, a flash memory, or the like. Further, the removable storage drive may read from and/or write to a removable storage device in a manner known in the art. In an embodiment, the removable storage unit may be a non-transitory computer readable recording media.

The computer system 500 may further include an I/O port 510 and a communication interface 512. The I/O port 510 may include various input and output devices that are configured to communicate with the processor 502. Examples of the input devices may include a keyboard, a mouse, a joystick, a touchscreen, a microphone, and the like. Examples of the output devices may include a display screen, a speaker, headphones, and the like. The communication interface 512 may be configured to allow data to be transferred between the computer system 500 and various devices that are communicatively coupled to the computer system 500. Examples of the communication interface 512 may include a modem, a network interface, i.e., an Ethernet card, a communications port, and the like. Data transferred via the communication interface 512 may be signals, such as electronic, electromagnetic, optical, or other signals as will be apparent to a person of ordinary skill in the art. The signals may travel via a communications channel, such as the communication network 114, which may be configured to transmit the signals to the various devices that are communicatively coupled to the computer system 500. Examples of the communication channel may include a wired, wireless, and/or optical medium such as cable, fiber optics, a phone line, a cellular phone link, a radio frequency link, and the like. The main memory 506 and the secondary memory 508 may refer to non-transitory computer readable mediums that may provide data that enables the computer system 500 to implement the methods illustrated in FIGS. 2A-2C and 3.

Various embodiments of the disclosure provide a non-transitory computer readable medium having stored thereon, computer executable instructions, which when executed by a computer, cause the computer to execute operations for sharing medical records of a user from a first entity to a second entity. The operations include receiving a data release request from the user to release medical records of the user. The operations further include determining whether a photo of the user is available in the medical records. The operations further include determining whether a medical application is installed on a communication device of the user. The operations further include sending a push notification to the communication device. The operations further include opening the medical application on the communication device upon receiving a trigger signal from the user. The operations further include activating an image capturing sensor of the communication device upon opening of the medical application. The operations further include capturing the photo of the user through the image capturing sensor. The operations further include determining whether the user is an authorized user by analyzing the captured photo. The operations further include sharing medical records of the user from the first entity to the second entity.

A person of ordinary skill in the art will appreciate that embodiments and exemplary scenarios of the disclosed subject matter may be practiced with various computer system configurations, including multi-core multiprocessor systems, minicomputers, mainframe computers, computers linked or clustered with distributed functions, as well as pervasive or miniature computers that may be embedded into virtually any device. Further, the operations may be described as a sequential process, however some of the operations may in fact be performed in parallel, concurrently, and/or in a distributed environment, and with program code stored locally or remotely for access by single or multiprocessor machines. In addition, in some embodiments, the order of operations may be rearranged without departing from the spirit of the disclosed subject matter.

Techniques consistent with the disclosure provide, among other features, systems and methods for sharing medical records of a user from a first entity to a second entity. While various exemplary embodiments of the disclosed systems and methods have been described above, it should be understood that they have been presented for purposes of example only, and not limitations. It is not exhaustive and does not limit the disclosure to the precise form disclosed. Modifications and variations are possible in light of the above teachings or may be acquired from practicing of the disclosure, without departing from the breadth or scope.

While various embodiments of the disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

It will finally be understood that the disclosed embodiments are presently preferred examples of how to make and use the claimed invention, and are intended to be explanatory rather than limiting of the scope of the invention as defined by the claims below. Reasonable variations and modifications of the illustrated examples in the foregoing written specification and drawings are possible without departing from the scope of the invention as defined in the claim below. It should further be understood that to the extent the term “invention” is used in the written specification, it is not to be construed as a limited term as to number of claimed or disclosed inventions or the scope of any such invention, but as a term which has long been conveniently and widely used to describe new and useful improvements in technology The scope of the invention supported by the above disclosure should accordingly be construed within the scope of what it teaches and suggests to those skilled in the art, and within the scope of any claims that the above disclosure supports. The scope of the invention is accordingly defined by the following claims.

Although particular embodiments of the invention have been described in detail for purposes of illustration, various modifications and enhancements may be made without departing from the spirit and scope of the invention.

What is claimed is:

1. A method for sharing medical records of a user from a first entity to a second entity, comprising:
receiving a data release request from the user to release medical records of the user;
determining whether a photo of the user is available in the medical records;
determining whether a medical application is installed on a communication device of the user;
sending a push notification to the communication device;
opening the medical application on the communication device upon receiving a trigger signal from the user;
activating an image capturing sensor of the communication device upon opening of the medical application;
capturing the photo of the user through the image capturing sensor;
determining whether the user is an authorized user by analyzing the captured photo; and
sharing medical records of the user from the first entity to the second entity.

2. The method of claim 1, wherein the data release request is received from the communication device of the user over a communication network.

3. The method of claim 1, wherein the medical records includes medical history of the user.

4. The method of claim 1, wherein the determining of whether the medical application is installed on the communication device of the user is performed when the photo of the user is available in the medical records.

5. The method of claim 1, wherein the push notification is sent to the communication device to perform capturing of an image of the user in order to verify the data release request.

6. The method of claim 5, wherein the image capturing sensor is a camera of the communication device.

7. The method of claim 1, wherein the medical records of the user are shared from the first entity to the second entity when the user is determined as the authorized user.

8. The method of claim 1, further comprising sending, when there is a match between the captured photo and a previously stored photo of the user, an authentication success message to the user and releasing the medical records of the user to the second entity.

9. The method of claim 1, further comprising sending, when there is no match between the captured photo and a previously stored photo of the user, an authentication unsuccess message to an administrator of a medical record sharing platform.

10. A system for sharing medical records of a user from a first entity to a second entity, the system comprising:
circuitry configured to:
receive a data release request from the user to release medical records of the user;
determine whether a photo of the user is available in the medical records;
determine whether a medical application is installed on a communication device of the user;
send a push notification to the communication device;
open the medical application on the communication device upon receiving a trigger signal from the user;
activate an image capturing sensor of the communication device upon opening of the medical application;
capture the photo of the user through the image capturing sensor;
determine whether the user is an authorized user by analyzing the captured photo; and
share medical records of the user from the first entity to the second entity.

11. The system of claim 10, wherein the circuitry is configured to receive the data release request from the communication device of the user over a communication network.

12. The system of claim 10, wherein the medical records includes medical history of the user.

13. The system of claim 10, wherein the determining of whether the medical application is installed on the communication device of the user is performed when the photo of the user is available in the medical records.

14. The system of claim 10, wherein the circuitry is configured to send the push notification to the communication device to perform capturing of an image of the user in order to verify the data release request.

15. The system of claim 14, wherein the image capturing sensor is a camera of the communication device.

16. The system of claim 10, wherein the medical records of the user are shared from the first entity to the second entity when the user is determined as the authorized user.

17. The system of claim 10, wherein the circuitry is further configured to send, when there is a match between the captured photo and a previously stored photo of the user, an authentication success message to the user and releasing the medical records of the user to the second entity.

18. The system of claim 10, wherein the circuitry is further configured to send, when there is no match between the captured photo and a previously stored photo of the user, an authentication unsuccess message to an administrator of a medical record sharing platform.
